(19) United States
(12) Patent Application Publication
(10) Pub. No.: US 2014/0272998 A1
(43) Pub. Date: Sep. 18, 2014

(54) DIAGNOSTIC MICRORNA PROFILING IN CUTANEOUS T-CELL LYMPHOMA (CTCL)

(71) Applicants: Ulrik Ralfkiaer, Kobenhavn O (DK); Peter Hagedorn, Horsholm (DK); Charlotte Busch Ahler, Tastrup (DK); Carsten Geisler, Kobenhavn K (DK); Anders Woetmann, Kobenhav N (DK); Lone Skov, Vedbaek (DK); Niels Feentved Odum, Kobenhavn K (DK); Elisabeth Ralfkiaer, Horsholm (DK)

(72) Inventors: Ulrik Ralfkiaer, Kobenhavn O (DK); Peter Hagedorn, Horsholm (DK); Charlotte Busch Ahler, Tastrup (DK); Carsten Geisler, Kobenhavn K (DK); Anders Woetmann, Kobenhav N (DK); Lone Skov, Vedbaek (DK); Niels Feentved Odum, Kobenhavn K (DK); Elisabeth Ralfkiaer, Horsholm (DK)

(73) Assignees: LEO PHARMA A/S, Ballerup (DK); COPENHAGEN UNIVERSITY, Copenhagen K (DK); RIGSHOSPITALET, Copenhagen Ø (DK); GENTOFTE HOSPITAL, Hellerup (DK)

(21) Appl. No.: 14/232,806

(22) PCT Filed: Jul. 13, 2012
(86) PCT No.: PCT/IB2012/001702

§ 371 Date: Jun. 3, 2014

Related U.S. Application Data
(60) Provisional application No. 61/508,231, filed on Jul. 15, 2011.
(60) Provisional application No. 61/508,838, filed on Jul. 18, 2011.

(51) Int. Cl. C12Q 1/68

(52) U.S. Cl. CPC C12Q 1/6886
USPC 435/6.12; 702/19

(57) ABSTRACT

The present invention relates to the field of cancer-diagnostics. In particular the invention relates to a microRNA expression signature that allows discriminating skin samples of cutaneous T-cell lymphomas (CTCL) from non-malignant (inflammantory) skin samples by use of quantitative polymerase chain reaction performed on reverse transcribed miRNA. miR-155, miR-326, miR-663b, miR-203 and miR-205 are shown to be differentially expressed.

### FIELD OF THE INVENTION

The present invention relates to the field of cancer-diagnostics. In particular the invention relates to a microRNA expression signature that allows discriminating non malignant (inflammantory) skin samples from skin samples of cutaneous T-cell lymphomas (CTCL).

### BACKGROUND OF THE INVENTION

Cutaneous T-cell lymphomas (CTCL) are the most frequent primary lymphomas of the skin, with mycosis fungoides (MF) being the most prevalent clinical form accounting for around 60% of new cases (Trautinger, 2006).

In early disease stages, which can last several years, MF presents as flat erythematous skin patches resembling inflammatory diseases such as dermatitis or psoriasis. In later stages, MF lesions gradually form plaques and overt tumors and may disseminate to lymph nodes and internal organs. The early skin lesions of this disease contain numerous inflammatory cells, including a large quantity of T cells with a normal phenotype as well as a small population of T cells with a malignant phenotype.

The infiltrate primarily consists of non-malignant T helper 1 (Th1) cells, regulatory T cells (Treg), and cytotoxic CD8⁺ T cells, which to some degree seem to control the malignant T cells (Lee, 1999; Gjerdrum, 2007). The malignant T cells typically exhibit the phenotype of mature CD4⁺ memory T cells and are normally of clonal origin (Rosen, 2006). T cells with a malignant phenotype are characterized by epidermotropism and are preferentially present in the upper parts of the skin, whereas T cells with a normal phenotype primarily are detected in the lower portions of the dermis. The epidermal T cells are sometimes found in patterns of Pautrier micro-abscesses, which are collections of T cells adherent to dendritic processes of Langerhans cells. During disease development, the epidermotropism is gradually lost concomitant with an increase in malignant, and a decrease in non-malignant, infiltrating T cells.

The etiology of CTCL remains poorly understood, and occupational exposures, infectious agents, and genetic mutations have been proposed as etiological factors, but no evidence of causation has been provided (Dereure, 2002). Instead, an aberrant expression and function of transcription factors and regulators of signal transduction is a characteristic feature of CTCL. Accordingly, it has been hypothesized that a dysfunctional regulation of signal molecules and cytokines plays a key role in the malignant transformation and epigenetic modifications such as aberrant gene methylation and histone de-acetylation are clearly involved in the pathogenesis of CTCL (Doorn, 2009; Girardi, 2004).

Early diagnosis is difficult because of the great clinical and histological resemblance to benign inflammatory diseases such as dermatitis or psoriasis. A definitive diagnosis from a skin biopsy requires the presence of convoluted lymphocytes, a band-like upper dermal infiltrate, and epidermal infiltrations with Pautrier abscesses, but all of these features are often not present in early stages and the histological picture is often difficult to interpret. Histological examinations can be supplemented by surrogate markers such as low CD7 and intermediate CD4 expression and cell receptor clonality, none of which are specific for CTCL. Moreover, these methods are laborious and no definitive disease markers exist. Accordingly, a definitive diagnosis may require the review of multiple biopsies over an extended period of time by an experienced pathologist. Thus, patients are often left in uncertainty for an extended period of time and subjected to different kinds of inefficient treatments.

MicroRNAs (miRNAs or miRs) are an abundant class of short endogenous RNAs that act as posttranscriptional regulators of gene expression by base-pairing with their target mRNAs. Specifically, miRNAs prevent mRNA translation and/or mediate target mRNA degradation. miRNAs are 19-25 nucleotide (nt) RNAs that are processed from longer endogenous hairpin transcripts (Ambros et al. 2003, RNA 9: 277-279). To date more than 6000 miRNAs have been identified in humans, worms, fruit flies and plants according to the miRNA registry database release 11.0 in April 2008, hosted by Sanger Institute, UK.

Recent data indicate that several miRNAs, are differentially expressed and possibly also involved in the pathogenesis of cancer (Garzon, 2009)¹². Thus, miR-21 expression is upregulated and appears to play a role in the regulation of apoptosis in malignant T cells obtained from patients with Sezary Syndrome (SS), a leukemic variant of CTCL. These findings are in keeping with studies in other cancers where miRNAs have been ascribed a key role in cancer development and metastasis. Indeed, specific miRNAs are directly involved in the malignant transformation as they can function as oncogenes and tumor suppressors (Krejsgaard, 2009).

Early diagnosis of CTCL has important consequences concerning therapeutic options and determination of prognosis. Unfortunately, early diagnosis of CTCL has proven difficult because of the great clinical, pathological, and histological resemblance to benign inflammatory skin diseases Accordingly, there is a need for an early and precise malignancy diagnosis which would allowing targeted treatment with established treatment modalities such as topical chemotherapy or phototherapy.

### SUMMARY OF THE INVENTION

The present invention provides a method based on a diagnostic miRNA classifier which allow a fast and accurate classification of skin specimens as being malignant or benign.

Accordingly, the invention pertains to a method for classifying a test skin cell sample from an individual with an inflammatory skin disease as cutaneous lymphomas comprising: detecting microRNA expression levels at least of one of miR-155 and miR-326, and at least of one of miR-203 and miR-205, calculating a clinical score (S) of the test cell sample based on a dataset comprising the expression levels of said microRNAs, and classifying the test cell sample as cutaneous lymphomas or not based on the value of the clinical score.

### DEFINITIONS

Prior to a discussion of the detailed embodiments of the invention is provided a definition of specific terms related to the main aspects and embodiments of the invention.

The terms “cutaneous lymphoma”, “cutaneous T-cell lymphoma, “mycosis fungoides” and “Sezary syndrome” are used and in accordance with the WHO-EORTC guidelines as described (Olsen, 2007; Willemze, 2005; Foss, 2011; Burg, 2005).

The terms “miR”, “miRNA” and “microRNA” are used synonymously and refer to a class of about 18-25 nucleotides (nt) long non-coding RNAs derived from endogenous genes. They are processed from longer (ca 75 nt) hairpin-like precursors termed pre-miRs. MicroRNAs assemble in complexes termed miRNPs and recognize their targets by antisense complementarity. If the microRNAs match 100% their target, i.e. the complementarity is complete, the target mRNA is cleaved, and the miR acts like a siRNA. If the match is incomplete, i.e. the complementarity is partial, then the translation of the target mRNA is blocked.

As used herein the terms “let-7b”, “miR-103”, “miR-155”, “miR-184”, “miR-191”, “miR-203”, “miR-205”, “miR-24”, “miR-299-5p”, “miR-326”, “miR-34b”, “miR-423-5p”, “miR-663b”, “miR-711” or “miR-718” refer to the human miR sequences found in miR registry database release 12.0 or later and hosted by Sanger Institute, UK as well as their animal equivalents. Except for miR-711 and miR-718, all mills are human miR sequences commonly referred to by the prefix “hsa-”,e.g. hsa-miR-155 refer to the human miR-155. miR-711 and miR-718 were not published until registry database release 14.0.

As used herein the term “detecting the level of a miR” refer to the quantification of said miR. One way of quantification is described in the Examples i.e. qRT-PCR. However, the miR may be quantified in a multitude of other ways e.g. by arrays, northern blots, dot blots, RN′ase protection assays, quantitative mass spectroscopy or various quantitative PCR-based methods such as the TaqMan assay or the UniRT assay used in the examples.

The term “expression”, as used herein, refers to the transcription and/or accumulation of RNA-molecules within a call.

In the present context the terms “level of expression of a miR” and “level of a miR” are used synonymously as a measure of the “amount of a specific miR” that is detected in the sample. The “amount of a specific miR” may be expressed in either absolute or relative measures and refers to values obtained by both quantitative, as well as qualitative methods. One particularly preferred measure of the “amount of a specific miR” is the Crossing point (Cp) value obtained by real-time qRT-PCR as described in the examples. Another preferred measure of the “amount of a specific miR” is the “threshold cycle value (Ct)” value likewise obtained by real-time qRT-PCR as described in the examples. The Cp and the Ct measures of the “amount of a specific miR” provide roughly similar measures, see Bustin, S. A. (ed.) A-Z of quantitative PCR, IUL Biotechnology Series 5 (2004) 882 pages. Whether to choose Cp or Ct is largely a matter of choice of the machine the assay tied to and performed on. If the amplification is performed in a LightCycler® 480 Real-Time PCR System using the Roche LC software the amount of a specific miR is expressed by the Cp. If the amplification is performed in Applied Biosystems ABI Prism 7900HT 384-well instrument using the software provided with it the amount of a specific miR is expressed by the Ct.

The term “level” designates relative as well as absolute amounts of the miRs referred to.

The terms“Q-PCR” or “q-PCR” refers to quantitative polymerase chain reaction. Q-PCR is highly sensitive method for quantifying the amounts of specific DNA (and RNA) species in a test sample. As quantification of RNA by the PCR technique requires that the RNA is reverse transcribed it is often referred to as “qRT-PCR” or “RT-Q-PCR” to indicate that quantitative PCR is used to quantify specific RNAs. A thorough treatise of the Q-PCR and qRT-PCRtechniques can be found in Bustin, S. A. (ed.) A-Z of quantitative PCR, IUL Biotechnology Series 5 (2004) 882 pages, which hereby is incorporated herein by reference.

“UniRT” is a novel Q-PCR method. The method is described in Example 4 and International Patent Application WO 2010/085966.

### DETAILED DISCLOSURE OF THE INVENTION

In the present study we used microarrays for an initial screening of miRNAs with a potential ability to distinguish between malignant (CTCL) and benign inflammatory skin disorders such as psoriasis, atopic dermatitis, and contact dermatitis. Five miRNAs (miR-203, miR-205, miR-326, miR-663b, and miR-711) were identified, which discriminated with high accuracy (>90%) between malignant and benign conditions in a total of 198 patients including an initial training set of 90 patients, a test set of 58 patients and an independent cohort of 50 patients, example 1, FIG. 1. Importantly, the expression pattern of four out of five miRNAs (miR-203, miR-205, miR-326 and miR-663b) was verified using qRT-PCR on RNA samples from 103 patients. Accordingly one embodiment of the present invention is a method for classifying a test skin cell sample from an individual with an inflammatory skin disease as cutaneous lymphomas comprising detecting microRNA (miR) expression levels of a selection of miRs comprising e.g. miR-326, miR-203, miR-205 and miR-663b in the test skin cell sample, calculating a clinical score (S) of the test cell sample based on a dataset comprising the expression levels of said microRNAs, and classifying the test cell sample as cutaneous lymphomas or not based on the value of the clinical score.

Recent studies on subpopulations of CTCL patients identified miR-155 as differentially expressed in Sezary Syndrome (SS) patients and advanced (tumor-stage) mycosis fungoides (MF) patients, respectively (Fits, 2011; Kester, 2011). Interestingly, we were only able to verify the miR-155 expression data by the qRT-PCR technique. However, with respect to miR-155 we were able to confirm and extend the findings of van Kester et al. Furthermore, our qRT-PCR results showed that miR-155 was one of the most significantly differentially expressed micorRNAs in CTCL.

Therefore, one embodiment of the present invention is a method wherein the levels of microRNA miR-155, miR-326, miR-203, miR-205 and miR-663b are detected and used to calculate a clinical score (S) of the test cell sample.

Using the nearest shrunken centroid algorithm on qRT-PCR data, miR-155, miR-203, and miR-205 were identified as the most discriminative set of miRNAs, see example 3. Accordingly, in one preferred embodiment of the present invention, the levels of miR-155 and at least one of miR-203 and miR-205 are detected and used to calculate a clinical score (S) of the test cell sample.

Example 3, FIG. 3B, show that the differential expression was also clearly confirmed for miR-203, miR-205, and miR-326 with P-values below 10⁻¹¹. Thus in one preferred embodiment of the present invention, the levels of microRNA miR-326 and at least one of miR-203 and miR-205 are detected and used to calculate a clinical score (S) of the test cell sample.

As miR-155 and miR-326 are the 2 most upregulated miRs in cutaneous lymphoma biopsies a further preferred embodiment of the present invention is an method comprising detecting the levels of miR-326 and at least one of miR-203 and miR-205 and used this data to calculate a clinical score (S) of the test cell sample.

In a still further embodiment of the present method the levels of microRNA miR-155, miR-326, miR-203 and miR-205 are detected and used to calculate a clinical score (S) of the test cell sample.

Cutaneous T-cell lymphomas (CTCL) are the most frequent primary lymphomas of the skin, therefore a further preferred embodiment of the present invention is a method wherein the method is used to differentiate benign test skin samples and test skin samples wherein the cutaneous lymphoma is cutaneous T-cell Lymphoma (CTCL).

According to the invention, the clinical score (S) may be calculated in a number of different ways. Importantly, when the score is calculated as the difference between the crossing point-value (Cp) determined by qRT-PCR of miR-155 and the average Cp of miR-203 and miR-205 the score was found to distinguish patients with CTCL from benign skin diseases with very high sensitivity, specificity, and classification accuracy (95%), see example 3, FIG. 7.

Thus, in one embodiment, the clinical score is calculated as a ratio of the expression level of miR-155 and the average expression levels of miR-203 and/or miR-205.

As seen in example 3 (FIG. 3B) differential expression was clearly confirmed for miR-203, miR-205, and miR-326 with P-values below 10⁻¹³. Accordingly, in another embodiment, the clinical score is calculated as a ratio of the expression level of miR-326 and the average expression levels of miR-203 and/or miR-205.

In an even further embodiment the clinical score is calculated as a ratio of the expression level of miR-155 and miR-326 relative to the average expression levels of miR-203 and/or miR-205.

MiR's may be quantified in a number of ways e.g. by arrays, northern blots, dot blots, RN'ase protection assays, quantitative mass spectroscopy or various quantitative PCR-based techniques.

The enzyme used in the PCR-technique is in most instances a temperature resistant DNA polymerase, thus in order to provide the necessary DNA template for the DNA polymerase to act on, RNA's are copied into their DNA complement by the action of a reverse transcriptase, before subjected to PCR. In the subsequent steps of the method the DNA-copies (often referred to as cDNA) is subjected to a quantitative PCR (q-PCR). The collective method of reverse transcribing the RNA in a sample an subsequently quantifying it by q-PCR is referred to quantitative reverse transcription polymerase chain reaction, or “qRT-PCR”. A thorough treatise of the Q-PCR and qRT-PCR techniques can be found in Bustin, S. A. (ed.) A-Z of quantitative PCR, IUL Biotechnology Series 5 (2004) 882 pages, which hereby is incorporated herein by reference.

Today, the by far most sensitive, specific and convenient technique for quantifying microRNA is the qRT-PCR technique. Therefore in the most preferred embodiment of the resent method the expression levels of the microRNAs are determined by qRT-PCR, and a preferred example of an qRT-PCR method is the UniRT-method described in Example 4. However, similar results were obtained using the ABI Taqman qRT-PCR assay described in example 6, FIG. 6.

While e.g. the ratios:

$$\frac{( level of miR 155 )}{( \frac{level of miR 203}{2} + \frac{level of miR 205}{2} )}$$

$$or$$

$$\frac{( level of miR 326 )}{( \frac{level of miR 203}{2} + \frac{level of miR 205}{2} )}$$

$$or$$

$$\frac{( ( levelofmiR 155 ) + ( levelofmiR 326 ) ) / 2}{( ( levelofmiR 203 ) + ( levelofmiR 205 ) ) / 2}$$

$$or$$

$$\frac{\begin{matrix}( ( levelofmiR 155 ) + ( levelofmiR 326 ) + \\ ( levelofmiR 663 b ) ) / 3\end{matrix}}{( ( levelofmiR 203 ) + ( levelofmiR 205 ) ) / 2}$$

all are useful estimators for S, the read-out from a typical real-time QPCR instrument is often the so-called Cp (crossing point)-value or the threshold cycle value (Ct) value both of which may be obtained by real-time qRT-PCR. Both the Ct- and the Cp-value is related to the level of e.g. a specific miR, by the relation:

(liniar)expressionlevelofmiRx˜2^{-CP(miRx)}

Wherein Cp(miRx) designates the Cp-readout from real-time QPCR instrument specifically detecting one specific miR called miRx. Examples describes such an assay in details.

Accordingly, when the Cp-values are used as quantifiers of miR-levels, the expression:

$$\frac{( level of miR 155 )}{( \frac{level of miR 203}{2} + \frac{level of miR 205}{2} )}$$

is equivalent to:

+Cp(miR155)−Cp(miR203)/2−Cp(miR205)/2

Note that the less miR in the sample the more cycles are to be run before crossing point or the threshold cycle is reached. I.e. the larger the Cp (or Ct) value the less miR is present in the sample.

Realizing that cutaneous lymphomas of the skin and in particularly cutaneous T-cell lymphomas (CTCL) are characterized by an increased level of expression of miR-155 (and miR-326) and a decreased expression of miR-203 and miR-205 relative to the level in normal or benign skin make it possible to formulate a wide range of estimators of the clinical score S. Logistic regression is a widely used method for generating best fit linear models of data. For instance the clinical score, S, may be calculated as follows:

S=X*C(miR-155)+Y*C(miR-205)+Z*C(miR-203),

wherein “C” is the threshold cycle value (Ct) or the crossing point value (Cp), and wherein X, Y, and Z are coefficients determined by linear regression, under the constraint that X+Y+Z=0, in order to minimize the classification error.

Likewise a clinical score, S, may be calculated as S=X*C(miR-326)+Y*C(miR-205)+Z*C(miR-203), wherein “C” is the threshold cycle value (Ct) or the crossing point value (Cp), and wherein X, Y, and Z are coefficients determined by linear regression, under the constraint that X+Y+Z=0, in order to minimize the classification error.

Or expressed in more general terms the clinical score may be calculated as: S=X*C(miR-155)+Y*C(miR-326)+Z*C(miR-663b)+W*C(miR-203)+Q*C(miR-205), wherein “C=” is the threshold cycle value (Ct) or the crossing point value (Cp), and wherein X, Y, Z, W, and Q are coefficients determined by linear regression, under the constraint that X+Y+Z+W+Q=0, in order to minimize the classification error.

As evidenced in Example 3 and illustrated in FIG. 4 one particularly useful estimator is S=C(miR-155)−C(miR-205)/2−C(miR-203)/2, wherein “C” is the threshold cycle value (Ct) or the crossing point value (Cp). Accordingly in the most preferred embodiment of the invention is the embodiment wherein the clinical score, S, is calculated as S=C(miR-155)−C(miR-205)/2−C(miR-203)/2, wherein “C” is the threshold cycle value (Ct).

Using this estimator for the clinical score it is possible to formulate threshold-values, see example 3 and FIG. 4 A.

Accordingly, in one embodiment of the invention the clinical score, S, is calculated as S═C(miR-155)−C(miR-205)/2−C(miR-203)/2; and “C” is the crossing point value (Cp) and wherein when the clinical score “S” is lower than about 6.5, in particular lower than 6.0, the test indicates that the test skin cell sample is Cutaneous T-cell Lymphoma, and wherein when the clinical score “S” is higher than about 6.5, in particular higher than about 7.0, the test indicates that the test skin-cell sample is benign.

Bearing on the same principles the a clinical score may be calculated as: S=C(miR-326)−C(miR-205)/2−C(miR-203)/2 wherein “C” is the threshold cycle value (Ct) or the crossing point value (Cp) or even S=C(miR-155)/3+C(miR-326)/3+C(miR-663b)/3−C(miR-205)/2−C(miR-203)/2 wherein “C” is the threshold, cycle value (Ct) or the crossing point value (Cp).

### LEGENDS

FIG. 1. Expression profiles in training set for highly significant miRNAs. We analyzed microarray measurements of 688 miRNAs in the training set of 90 samples with t-test to discover differences in expression between samples of subjects with CTCL and those of benign inflammatory skin diseases or healthy individuals (BDN) subjects. The 27 miRNAs that displayed highly significant (Bonferroni corrected P<0.001) and strong differences (at least 50% change) are presented in the heat map. Samples are arranged in columns, miRNAs in rows, and both are hierarchically clustered using Euclidean distance with average linkage of nodes. Black-to white shades indicate increased relative expression; Black shades indicate reduced expression; gray indicates median expression. Top 5 most significantly induced or repressed microRNAs are shown in bold.

FIG. 2. Classification of CTCL and BDN. A. Principal component analysis (PCA) plot of samples from subjects with CTCL (light gray) and those of BDN subjects (dark gray) in the training set based on the 5-microRNA profile identified by the nearest shrunken centroids (NSC) algorithm. Percentages indicate percent variance explained by that component. B. Classification performance in the training set using the NSC algorithm. P-values were calculated using Fisher's exact test. C. PCA plot of samples in the test set based on the 5-microRNA profile identified from the training set. D. Classification performance in the test set using the trained NSC algorithm.

FIG. 3. Classifier miRNA expressions measured by microarray and qRT-PCR. For each microRNA, expressions are grouped according to patient type (CTCL and BDN respectively), with a small scatter on the x-axis within each group to allow better visualization of all measurements. P-values were calculated using t-test. A. Expressions measured by microarray. B. Expressions measured by qRT-PCR.

FIG. 4. qRT-PCR-based classification of samples from patients with CTCL and benign skin disease. A. A Cp based sample score (S) were calculated for each sample. Patients are ordered by increasing values of this score. The solid line shows the cutoff between patients with CTCL (light gray) and patients with benign skin disease (dark gray). The dotted lines show the cutoffs for the low confidence region. B. Classification performance using the cutoffs defined in (A). P-values were calculated using Fisher's exact test. C. Receiver operator characteristic curve (ROC) showing the sensitivity and specificity for various cutoff values on the sample score of the samples. D. Relative expression of the three microRNAs used in the classification in samples from patients with CTCL and benign skin disease. Error bars indicate ±1 standard deviation. The dCp (or ΔCp) value is calculated as ΔCp=Cp, reference(=control)−Cp, observed.

FIG. 5. qRT-PCR-based classification of samples from patients with Mycosis Fungoides (MF) in various stages of the disease. A Cp based sample score (S) were calculated for each sample as in FIG. 4. Patients are ordered according to clinical stages (I to IV) and solid lines indicate mean sample score in each stage. Patients with a sample score below 6.52 are being classified as CTCL (c.f. FIG. 4).

FIG. 6. The two step UniRT protocol. The principle the qRT-PCR of a microRNA serves as an example, the RNA to be analysed by the method may as well be any other small RNA molecule or even a mRNA. Step 1 is a one-tube-reaction for all microRNAs present in a sample. Step 2 is a microRNA specific qPCR using forward and reverse primer pairs for a specific microRNA. An oval indicate insertion of Locked Nucleic Acids (LNAs) in forward and reverse primers. When the method is carried out in practice the miRNAs present in a sample are firstly poly-A-tailed using a poly(A) polymerase (SEQ ID NO: 47), which adds adenine residues to the 3′-end of RNA molecules. Secondly, an extension primer (SEQ ID NO: 48), which has a poly-T-core nucleotide sequence, a 3′-end VN- or VNN-degenerate motif and a 5′-end tail, is annealed to the poly-A-tailed miRNA through hybridisation with the VN- or VNN-poly-T-sequence of the extension primer, (N=C, G, A and T; V=C, G, and A). This primer may be referred to as the Universal RT primer. Subsequently, the extension primer is extended in a reverse transcription reaction using the miRNA as template. All of these reactions are performed in a one-tube reaction. The resulting primary extension product is composed of the extension primer and the newly synthesized DNA, which is cDNA complementary to all the miRNAs in the sample. In the next step a miRNA-specific PCR is carried out. A miRNA-specific forward primer is annealed to 3′-end of the newly synthesized cDNA and the upper-strand synthesis is carried out by extending the forward primer in a DNA-polymerization reaction using the primary extension product as template. A miRNA-specific reverse primer (SEQ ID NO: 49) composed of a miRNA-specific 3′-end sequence, a poly-T-stretch and a 5′-end tail is then hybridized to the upper-strand and the lower-strand is synthesized by extension of the reverse primer.

FIG. 7. Expressions measured by qRT-PCR for the microRNAs in the classifier. For each microRNA, expressions are grouped according to patient type (BDN and CTCL respectively). Within each group, there is a small scatter on the x-axis to allow better visualization of all measurements.

### EXPERIMENTAL

Microarray Data Preprocessing

Probe signals were background corrected by fitting a convolution of normal and exponential distributions to the foreground intensities using the background intensities as a covariate (Ritchie, 2007). Four technical replicate spots for each probe were combined to produce one signal by taking the logarithmic base-2 mean of reliable spots. If all four replicates for a given probe were judged unreliable that probe was removed from further analysis. A reference data vector R was calculated as the median signal of each probe across all samples. For all probe signals in a given sample, represented by the sample data vector S, a curve F was determined by locally weighted polynomial regression so as to provide the best fit between S and R (Cleveland, 1992). A normalized sample vector M was calculated from this by transforming it with the function F, so that M=F(S). In this manner, all samples were normalized to the reference R. This normalization procedure largely follows that outlined in (Rosenfeld, 2008). Remote data points (probes in sparsely sampled intensity regions with less than 15 probes per signal unit) were considered unreliably adjusted by this method and removed before further analysis.

Classifier Statistics

Significance of differences in expression levels was assessed by a two-sided unpaired t-test. Class prediction was done using nearest shrunken centroid classification (Tibshirani. 2002). Briefly, a standardized centroid is computed for each class as the average expression of each microRNA in each class divided by the within-class standard deviation for that microRNA. The microRNA expression profile of a new sample is then compared to each of these class centroids, and the class, whose centroid is closest in Euclidean distance, is the predicted class for that new sample. The algorithm is trained by shrinking class centroids towards the overall centroid for all classes by a threshold amount that minimizes the misclassification error as determined through 10-fold cross validation on the training set.

### Example 1

### miRNA Expression Profiling Using Microarrays

Microarray analyses were used to perform miRNA profiling of 148 formalin-fixed and paraffin-embedded biopsies. Of these samples, 63 were from patients with various forms of CTCL and 85 were from patients with benign inflammatory skin diseases or healthy individuals (BDN) (Table 1).

Biopsies from the lymphoma patients were sampled during the period 1979-2004 and were collected from the archives at the Departments of Pathology at Rigshospitalet, Bispebjerg Hospital, Aalborg Sygehus and Herlev Hospital. From all lymphoma cases, tissue samples were reviewed by histology and immunohistochemistry, using as a minimum CD3, CD4, CD8, CD30, CD56, TIA-1 and Granzyme B stains. The samples were then classified in accordance with the WHO-EORTC (Olsen, 2007; Willemze, 2005; Foss, 2011; Burg, 2005) guidelines and the clinical characteristics of the cohort were reviewed to establish the final diagnoses. Biopsies from the patients with benign skin diseases and healthy controls were collected after informed consent at the Department of Dermato-Allergology, Gentofte Hospital Department of Dermatology, Bispebjerg Hospital, Department of Pathology, Rigshospitalet, and as part of clinical trials at LEO Pharma A/S and approved by the local ethical committees (H-B-2009-045 and H-1-2009-111) and the Data Protection Agency (Datatilsynet J. N R. 201041-4303)

Total RNA was isolated from six 10 μm tissue sections using the RecoverAll Total Nucleic Acid Isolation Kit (Applied Biosystems/Ambion, USA) according to manufacturer guidelines. Total RNA quantity and quality were checked by spectrophotometer (Nanodrop ND-1000).

From each sample 100 ng of total RNA was labeled with Hy3 fluorescent dye using the miRCURY LNA Array power labeling kit (Exiqon, Denmark). All samples were labeled the same day with the same master mix, in order to minimize technical variation. The Hy3-labelled samples were hybridized to miRCURY LNA arrays (v11.0) (Exiqon, Denmark), containing capture probes targeting all human miRNAs registered in the miRBASE version at the Sanger Institute. The hybridization was performed overnight at 56° C. according to manufacturer specifications using a Tecan HS4800 hybridization station (Tecan, Austria). Since it was not possible to hybridize all arrays in one go, samples were randomly split into 5 batches as to minimize day-to-day variation in the hybridization process. After hybridization the microarray slides were scanned using an Agilent G2565BA Microarray Scanner System (Agilent Technologies, Inc., USA) at 5 μm resolution, and the resulting TIFF images were analyzed using the ImaGene 8.0 software on standard settings (BioDiscovery, Inc., USA).

Probe signals were background corrected by fitting a convolution of normal and exponential distributions to the foreground intensities using the background intensities as a covariate (Ritchie, 2007). Four technical replicate spots for each probe were combined to produce one signal by taking the logarithmic base-2 mean of reliable spots. If all four replicates for a given probe were judged unreliable that probe was removed from further analysis. A reference data vector R was calculated as the median signal of each probe across all samples. For all probe signals in a given sample, represented by the sample data vector S, a curve F was determined by locally weighted polynomial regression so as to provide, the best fit between S and R (Cleveland, 1992; Rosenfeld, 2008). A normalized sample vector M was calculated from this by transforming it with the function F, so that M=F(S). In this manner, all samples were normalized to the reference R. This normalization procedure largely follows that outlined in (Rosenfeld, 2008). Remote data points (probes in sparsely sampled intensity regions with less than 15 probes per signal unit) were considered unreliably adjusted by this method and removed before further analysis.

The samples were divided into 3/5 for training (n=90) and 2/5 for testing (n=58) with approximately equal proportion of CTCL to BDN samples in both sets. This division follows the five microarray production batches used in the study (Table 1). Out of the 688 miRNAs that passed preprocessing filtering criteria, initial statistical analysis of the training set identified 27 miRNAs showing strong (at least 50% change) and highly significant (Bonferroni corrected P-values <0.001 from t-test) differences between CTCL and benign skin diseases and normal skin (FIG. 1). Thus, the expression levels of a large number of miRNAs differ considerably between patients with CTCL and patients with BDN. Essentially similar results were obtained using unsupervised hierarchical clustering based on the 209 most variable miRNAs (data not shown).

TABLE 1
Clinical characteristics of patients in the study. ndications are stratified according to age, gender, and the
microarray production batch. P-values were calculated using Fisher's exact test on sums across sub-indications
(the □ columns). MF, mycosis fungoides; SS, sezary syndrome; CALCL, cutaneous anaplastic large cell
lymphoma; NOS, cutaneous T-cell lymphoma - not otherwise specified; AD, atopic dermatitis; ND,
unspecified dermatosis; PP, lesional skin from psoriasis patients; PN, non-lesional skin from psoriasis
patients; NN, normal skin from healthy controls.

| | Cutaneous Lymphoma (n = 63) | | | | | Benign skin disease or normal skin (n = 85) | | | | | | |
|---|---|---|---|---|---|---|---|---|---|---|---|---|
| | MF | SS | CALCL | NOS | | AD | ND | PP | PN | NN | | |
| | (n = 39) | (n = 7) | (n = 8) | (n = 9) | Σ | (n = 20) | (n = 4) | (n = 42) | (n = 17) | (n = 2) | Σ | P-Value |
| Age (years) * | | | | | | | | | | | | |
| <30 | 0 | 0 | 1 | 0 | 1 | 19 | 0 | 4 | 4 | 2 | 29 | <0.001 |
| 30-44 | 5 | 0 | 0 | 0 | 5 | 1 | 0 | 6 | 3 | 0 | 10 | |
| 45-59 | 9 | 1 | 1 | 2 | 13 | 0 | 1 | 19 | 7 | 0 | 27 | |
| 60-74 | 14 | 6 | 2 | 2 | 24 | 0 | 3 | 12 | 2 | 0 | 17 | |
| ≧75 | 10 | 0 | 2 | 5 | 17 | 0 | 0 | 1 | 1 | 0 | 2 | |
| Gender * | | | | | | | | | | | | |
| Male | 23 | 7 | 5 | 5 | 40 | 8 | 1 | 30 | 16 | 2 | 57 | 1.00 |
| Female | 15 | 0 | 1 | 4 | 20 | 12 | 3 | 12 | 1 | 0 | 28 | |
| Microarray batch | | | | | | | | | | | | |
| 1 | 8 | 2 | 1 | 2 | 13 | 4 | 1 | 8 | 3 | 1 | 17 | 1.00 |
| 2 | 7 | 2 | 2 | 1 | 12 | 4 | 1 | 8 | 3 | 1 | 17 | |
| 3 | 7 | 1 | 2 | 2 | 12 | 4 | 1 | 8 | 3 | 0 | 16 | |
| 4 | 9 | 1 | 2 | 2 | 14 | 4 | 0 | 9 | 4 | 0 | 17 | |
| 5 | 8 | 1 | 1 | 2 | 12 | 4 | 1 | 9 | 4 | 0 | 18 | |

* Gender and age of 2 CALCL samples and one MF sample are unknown

### Example 2

### Identification of a CTCL—Specific miRNA Signature

To find a CTCL-specific signature we analyzed the training set with a nearest shrunken centroid algorithm (Tibshirani, 2002). The top 3 most induced (miR-326, miR-663b, miR-711) and 2 most repressed (miR-203, miR-205) miRNAs among the 27 highly significant miRNAs identified in the array analysis were found to be the optimal set of miRNAs for classification after shrinkage of centroids.

Significance of differences in expression levels was assessed by a two-sided unpaired t-test. Class prediction was done using nearest shrunken centroid classification (Tibshirani, 2002). Briefly, a standardized centroid is computed for each class as the average expression of each microRNA in each class divided by the within-class standard deviation for that microRNA. The microRNA expression profile of a new sample is then compared to each of these class centroids, and the class, whose centroid is closest in Euclidean distance, is the predicted class for that new sample. The algorithm is trained by shrinking class centroids towards the overall centroid for all classes by a threshold amount that minimizes the misclassification error as determined through 10-fold cross validation on the training set.

All five miRNAs had Bonferroni corrected P-values <10⁻⁸ by t-test. Samples in the training set could be classified with 93% accuracy (84% sensitivity and 100% specificity, P<0.001 by Fisher's exact test, (FIG. 2). To assess the performance of the five miRNAs in the classification of unknown samples, we used the already trained classifier on the 59 test set samples, which were classified with 97% classification accuracy (92% sensitivity and 100% specificity, P<0.001 by Fisher's exact test) (FIGS. 2C and 2D). FIG. 3A shows the expression of the individual miRNAs in the classifier. For each of the five miRNAs, the normalized log 2 expression values are grouped according to patient type (CTCL and BDN, respectively) (FIG. 3A).

Next, we evaluated the robustness of the classifier by ten-fold cross-validation, each time selecting different batches as training and test set (but keeping the ratio 3/5 to 2/5). The above approach identified miR-203, miR-663b, miR-205, and miR-711 in almost all cases (Table 2). One miRNA, miR-326, was only selected in 2 out of 10 divisions. Importantly, no matter which division was chosen, the classification accuracy in the test set was consistently above 90% (93.1% in average with a 99% confidence interval between 90.5% and 95.6%) (Table 2).

TABLE 2
Evaluation of robustness of classification. The five microarray production batches can be divided into 3/5 training
and 2/5 test sets in 10 different ways. For each division, miRNAs were selected and a classifier trained.
The classifier reported in this study corresponds to the one trained in round two.

| | Batches selected | n test | n test | Batches selected | n | n | Accuracy | Accuracy | |
|---|---|---|---|---|---|---|---|---|---|
| Round | for test set | (CTCL) | (BDN) | for training set | (CTCL) | (BDN) | (train) | (test) | miRNAs in classifier |
| 1 | 1, 2 | 25 | 34 | 3, 4, 5 | 38 | 51 | 0.94 | 0.95 | 203, 205, 299-5p, 663b, 711, 718 |
| 2 | 1, 3 | 25 | 33 | 2, 4, 5 | 38 | 52 | 0.93 | 0.97 | 203, 205, 326, 663b, 711 |
| 3 | 1, 4 | 27 | 34 | 2, 3, 5 | 36 | 51 | 0.95 | 0.89 | 203, 326, 663b, 718, 1252, 1249 |
| 4 | 1, 5 | 25 | 35 | 2, 3, 4 | 38 | 50 | 0.95 | 0.92 | 203, 205, 663b, 711, 718, 1252 |
| 5 | 2, 3 | 24 | 33 | 1, 4, 5 | 39 | 52 | 0.91 | 0.98 | 203, 205, 299-5p, 663b, 711 |
| 6 | 2, 4 | 26 | 34 | 1, 3, 5 | 37 | 51 | 0.95 | 0.92 | 34b, 203, 663b, 1249, 1252 |
| 7 | 2, 5 | 24 | 35 | 1, 3, 4 | 39 | 50 | 0.94 | 0.93 | 34b, 203, 205, 299-5p, 663b, 1249 |
| 8 | 3, 4 | 26 | 33 | 1, 2, 5 | 37 | 52 | 0.93 | 0.94 | 203, 205, 663b, 665, 1252 |
| 9 | 3, 5 | 24 | 34 | 1, 2, 4 | 39 | 51 | 0.93 | 0.93 | 203, 205, 663b, 711, 1285 |
| 10 | 4, 5 | 26 | 35 | 1, 2, 3 | 37 | 50 | 0.97 | 0.89 | 203, 490-3p, 663b, 1249, 1252 |

### Example 3

### Identification of a qRT-PCR-Based miRNA Classifier

For diagnostic purposes, qRT-PCR is more sensitive, specific, and applicable than microarrays. To confirm the microarray results above, expression levels were measured by qRT-PCR for the 5-miRNA signature on a subset of 103 samples of the 148 samples described in example 1. The subset of samples were selected based on high RNA content as measured by NanoDrop and covered both training and test set samples.

Total RNA was isolated from six 10 μm tissue sections using the RecoverAll Total Nucleic Acid Isolation Kit (Applied Biosystems/Ambion, USA) according to manufacturer guidelines. Total RNA quantity and quality were checked by spectrophotometer (Nanodrop ND-1000). cDNA was diluted 50× and assayed in 10 μl PCR reactions according to the protocol for miRCURY LNA™ Universal RT microRNA PCR; each microRNA was assayed once by qPCR. Negative controls excluding template from the reverse transcription reaction were performed and profiled in parallel. The amplification was performed in a LightCycler® 480 Real-Time PCR System (Roche) in 384 well plates. The amplification curves were analyzed using the Roche. LC software, both for determination of Cp (by the 2nd derivative method) and for melting curve analysis. All assays were inspected for distinct melting curves and the Tm was checked to be within known specifications for the assay. Furthermore, assays must be detected with 3 Cp's less than the negative control, and with Cp<39 to be included in the data analysis. Data that did not pass these criteria were omitted from any further analysis.

MiR-103 and miR-423-5p were identified as the most stably expressed references across, samples and their average Cp, denoted Cp,ref used as normalization factor when calculating ΔCp (Vandesompele, 2002). Specifically, for the Cp measured from a given miRNA, the ΔCp (or dCp) value is calculated as ΔCp=Cp,ref−Cp, obs. The differential expression was clearly confirmed for miR-203, miR-205, and miR-326 with P-values below 10¹¹ and for miR-663b with a P-value below 10⁻⁷ (FIG. 3B) whereas the last miRNA, miR-711 could not be measured reliably above background fluorescence. Essentially similar results were obtained in an independent series of qRT-PCR experiments on 44 patient samples using a different qRT-PCR platform (TaqMan-data not shown). Recent studies on skin lesions from tumor stage MF and blood samples from SS patients reported on a differential expression of miR-155, miR-21, miR-24, miR-34b, miR-191, miR-486, Let-7b, and other miRNAs (Chen, 2010; Ballabio, 2010; Holst, 2010; Narducci, 2011).

Accordingly, we performed qRT-PCR for these miRNAs and confirmed differential expression of miR-155, miR-24, miR-191, and Let-7b. In contrast, miR-34b did not achieve significance in the qRT-PCR measurements whereas miR-21 was increased in CTCL but also in a fraction of psoriasis patients (data not shown). The nearest shrunken centroid algorithm identified miR-155, miR-203, and miR-205 as the most discriminative set of miRNAs. By rewriting the equation for nearest centroid classification as an equivalent linear combination (Richard O. Duda, Peter E. Hart, David G. Stork, “Pattern Classification”, Wiley Interscience, 2^{nd} edition, 2001, pages 36-39), we obtained a simplified discriminant function, or sample score, as: S=Cp(miR-155)−Cp(miR-203)/2−Cp(miR-205)/2.

The area under the receiver operator characteristic curve (ROC, FIG. 4C) was 0.989 with 95% confidence interval between 0.9725 and 0.9996 (as calculated from 10000 stratified bootstrap replicates, Carpenter and Bithell, 2000). The significance of this result was estimated by the Wilcoxon rank-sum test to be P<2×10⁻¹⁶.

We chose thresholds by inspecting the distribution of sample scores (FIG. 4A+B) and the ROC curve (FIG. 4C), and introduced a low-confidence region around the threshold (FIG. 4A+B). As miR-203 and 205 expression was decreased in CTCL (FIG. 4D) and miR-155 expression increased in CTCL (FIG. 4D), the score S was smaller in CTCL when compared to benign skin disorders (FIG. 4A+B). In 103 samples, this qRT-PCR-based “minimal” miRNA classifier (miR-155, miR-203, and miR-205) distinguished patients with CTCL from benign skin diseases with 95% classification accuracy (P<0.001, FIG. 4B) and high sensitivity/specificity as illustrated by the ROC graph in FIG. 4C (dot indicates 91% sensitivity at 97% specificity). Importantly, MF patients were classified as malignant independently of the disease stage (FIG. 5). It is noteworthy that the signature differentiate even between early stages of MF and benign controls (BDN).

The sensitivity and specificity achieved by our miRNA classifier constitutes a significant improvement compared to current practice. In addition, the highly significant ROC curve, with an area under the curve (AUC) very close to 1, makes it highly plausible that these classification accuracies will extend to new samples.

### Example 4

### The UniRT Method

In this example the UniRT method for amplification and quantification of small non-coding RNA molecules by use of quantitative reverse transcription polymerase chain reaction (qRT-PCR) technology is described in brief.

In brief, see FIG. 6, the UniRT protocol is a two-step protocol. In STEP 1 the miRs present in a sample are firstly poly-A-tailed using a poly(A) polymerase, which adds adenine residues to the 3′-end of RNA molecules. Secondly, an extension primer, which has a poly-T-core nucleotide sequence, a 3′-end VN-degenerate motif and a 5′-end tail, is annealed to the poly-A-tailed miRs through hybridization with the VN-poly-T-sequence of the extension primer. Subsequently, the extension primer is extended in a reverse transcription reaction using the miR as template. The resulting primary extension product, is composed of the extension primer and the newly synthesized cDNA, which complementary to the miRs in the sample.

In the next step, STEP 2, a miR-specific PCR is carried out A miR-specific forward primer is annealed to 3′-end of the newly synthesized cDNA and the upper-strand synthesis is carried out by extending the forward primer in a DNA-polymerization reaction using the primary extension product as template. A miR-specific reverse primer composed of a miR-specific 3′-end sequence, a poly-T-stretch and a 5′-end tail is then hybridized to the upper-strand and the lower-strand is synthesized by extension of the reverse primer.

In both STEP 1 and STEP 2 the LNA's help to ensure a specific and efficient annealing of the primers to their respective targets.

### Example 5

### Identification of microRNAs by the UniRT Method

The levels of let-7b, miR-103, miR-155, miR-184, miR-191, miR-203, miR-205, miR-299-5p, miR-326, miR-423-5p, miR-663b, miR-711 and miR-718 were quantified using the UniRT qPCR method (see Example 4). In brief,

In STEP 1 of the UniRT protocol 10 ng of total RNA was used per 10 μl RT reaction having the composition:
Reaction buffer (1× Reaction buffer contains; 167 mM NaCl, 25 mM KCl, 50 mM Tris-HCl, 8 mM MgCl2, 3.33 mM DTT, 0.1 mM ATP, 0.1 mM dATP, 0.1 mM dCTP, 0.1 mM dGTP and 0.1 mM dTTP)
0.5 μM RT-primer (L2TA3: 5′-ggtactagtttttttttttttttvnn (SEQ ID NO. 1)), or (v designates cytosine, guanine and adenine residues, n designates cytosine, guanine, adenine and thymine residues).
100 unit of Moloney Murine Leukemia Virus (M-MuLV) Reverse Transcriptase (New England Biolabs, Ipswich Mass.)
1 unit of E. coli Poly (A) Polymerase. (New England Biolabs, Ipswich Mass.)
The RT reactions were run in triplicate (three RT reactions per sample).
The RT reaction was incubated at 42° C. for 1 hour, 95° C. for 5 minutes.
Then the RT reaction was diluted 50× in water prior to qPCR analysis—STEP 2.

In STEP 2 of the UniRT protocol 1 μl of the diluted RT reaction was mixed with the PCR primer sets of Table (final concentration of each primer is 0.3 μM) for each miR and Fast start SYBR Green Master mix (Roche Diagnostics GmbH, Mannheim, Germany) according to protocol by the providers.

All qPCRs were run in singlicates (one qPCR reaction per. RT reaction) in 10 μl reaction volume. The qPCR reactions were run in 384 well plates, in a Roche Lightcycler 480 II (Roche Diagnostics GmbH, Mannheim, Germany).

All real-time PCR data were analyzed using the Cp (Crossing point) method calculating the relative expression ratios of the specific target miRs as the crossing point difference (ΔCp) of the specific miRs relative to one or several reference genes (Bustin.2004; Vandesompele, 2002).

The Cp-values were calculated using the Lightcycler 480 software release 1.5.0, version 1.5.0.39 accompanying the Lightcycler 480 II instrument.

TABLE 3
microRNA and Primer sequences

| | miRBase | | F primers_Primer | R primers_Primer |
|---|---|---|---|---|
| | version | miR sequence (5′ → 3′) | sequence (5′ → 3′) | sequence (5′ → 3′) |
| let- | 12 | tgaggtagtaggttgtgtggtt | catgaggtagtaggttg | ggtactagtttttttttttttttaaccac |
| 7b | | (SEQ ID NO. 2) | (SEQ ID NO. 17) | (SEQ ID NO. 32) |
| miR- | 12 | agcagcattgtacagggctatga | agcagcattgtacagg | gtactagtttttttttttttttcatagc |
| 103 | | (SEQ ID NO. 3) | (SEQ ID NO. 18) | (SEQ ID NO. 33) |
| miR- | 12 | ttaatgctaatcgtgataggggt | gacttaatgctaatcgtgat | gtactagtttttttttttttttaccccta |
| 155 | | (SEQ ID NO. 4) | (SEQ ID NO. 19) | (SEQ ID NO. 34) |
| miR- | 12 | tggacggagaactgataagggt | tggacggagaactgat | gtactagtttttttttttttttaccct |
| 184 | | (SEQ ID NO. 5) | (SEQ ID NO. 20) | (SEQ ID NO. 35) |
| miR- | 12 | caacggaatcccaaaagcagctg | caacggaatcccaaaagc | gtactagtttttttttttttttcagc |
| 191 | | (SEQ ID NO. 6) | (SEQ ID NO. 21) | (SEQ ID NO. 36) |
| miR- | 12 | gtgaaatgtttaggaccactag | gtgaaatgtttaggacca | tgacacggaggtactagtttttttttttt |
| 203 | | (SEQ ID NO. 7) | (SEQ ID NO. 22) | tttctag |
| | | | | (SEQ ID NO. 37) |
| miR- | 12 | tccttcattccaccggagtctg | tccttcattccaccgga | gtactagtttttttttttttttcagact |
| 205 | | (SEQ ID NO. 8) | (SEQ ID NO. 23) | (SEQ ID NO. 38) |
| miR- | 12 | tggctcagttcagcaggaacag | tggctcagttcagca | tgacacggaggtactagtttttttttttttt |
| 24 | | (SEQ ID NO. 9) | (SEQ ID NO. 24) | ctgttc |
| | | | | (SEQ ID NO. 39) |
| miR- | 12 | tggtttaccgtcccacatacat | tggtttaccgtcccacat | gaggtactagtttttttttttttttatgta |
| 299-5p | | (SEQ ID NO. 10) | (SEQ ID NO. 25) | (SEQ ID NO. 40) |
| miR- | 12 | cctctgggcccttcctccag | cctctgggcccttcct | gtactagtttttttttttttttctgga |
| 326 | | (SEQ ID NO. 11) | (SEQ ID NO. 26) | (SEQ ID NO. 41) |
| miR- | 12 | caatcactaactccactgccat | caatcactaactccactg | ggtactagtttttttttttttttatggc |
| 34b | | (SEQ ID NO. 12) | (SEQ ID NO. 27) | (SEQ ID NO. 42) |
| miR- | 12 | tgaggggcagagagcgagacttt | catgggcagagagc | aggtactagttttttttttttttaaagtc |
| 423-5p | | (SEQ ID NO. 13) | (SEQ ID NO. 28) | (SEQ ID NO. 43) |
| miR- | 12 | ggtggcccggccgtgcctgagg | ccggccgtgcct | gtactagtttttttttttttttcctca |
| 663b | | (SEQ ID NO. 14) | (SEQ ID NO. 29) | (SEQ ID NO. 44) |
| miR- | 14 | gggacccagggagagacgtaag | agggacccagggaga | ggtactagtttttttttttttttcttacg |
| 711 | | (SEQ ID NO. 15) | (SEQ ID NO. 30) | (SEQ ID NO. 45) |
| miR- | 14 | cttccgccccgccgggcgtcg | tatcttccgccccgccg | ttttttttcgacgc |
| 718 | | (SEQ ID NO. 16) | (SEQ ID NO. 31) | (SEQ ID NO. 46) |

Primer sequences are spiked with LNA (0% to 13.3% of the nucleotides) in combination with natural occurring nucleotides. Except for miR-711 and miR-718, all miRs are human miR sequences found in miR registry database release 12.0 hosted by Sanger Institute, UK (miR 12_0) miR-711 and miR-718 were not published until registry database release 14.0.

### Example 6

### ABI TaqMan qRT-PCR Assay

Skin biopsy samples were obtained from patients with cutaneous T-cell lymphoma, patients with benign skin diseases including psoriasis and atopic dermatitis, and healthy subjects. FFPE blocks were sectioned in RNase free environment. Eight 10 μm tissue sections were placed in each of two 1.5 mL microcentrifuge tubes (16 sections in total) and total RNA extracted using the RecoverAll Total Nucleic Acid Isolation Kit (Applied Biosystems/Ambion, USA) according to manufacturer guidelines. From each sample 15 ng of total RNA was reverse transcribed to cDNA using the TaqMan Micro RNA Reverse Transcription. Kit with appropriate primers (Applied Biosystems, USA) following manufacturer guidelines. Triplicates of 5 ng cDNA per reaction was each mixed with TaqMan Universal PCR Master Mix and the relevant primers and probe (Applied Biosystems, USA) and run on the Applied Biosystems-ABI 79001-IT 384-well instrument on standard settings. The cycle threshold (Ct, the PCR cycle at which probe signal reaches a threshold value above fluorescent background) was determined for each well.

Measurements of Ct from three technical replicate wells for each probe were combined to produce one signal by taking the mean of reliable wells. To allow identification of a stably expressed normalization factor, five commonly used small RNA references (RNU6A, RNU6B, RNU14B, RNU48 and SNORD12) were included besides the microRNAs of interest, and ranked according to their feature stability-measure across all samples (Vandesompele et al., 2002).

The two small RNAs RNU6B and SNORD12 were identified as the most stably expressed references across samples and their average Ct used as normalization factor when calculating ΔCt (Vandesompele et al., 2002). Samples were selected based on high RNA content and covered both training and test set samples. Differential expression was clearly confirmed for miR-203, miR-205, and miR-326 (Table 4).

TABLE 4
Expressions and significance for the 5 microRNAs in the classifier. Results are shown for both microarray
and qRT-PCR measurement. The Wilcox-test-on-ranks column reports P-values from a Wilcox test
performed on an un-normalized data matrix where intensities measured on each array are replaced by
their rank when comparing with all intensities on that array. Significance from this test therefore indicates
that the intensity for that microRNA clearly changes rank between the conditions compared (being for
example among the lowest intensities on each array in one group of samples, and therefore having
consistently low rank, but among the highest intensities on each array in another group of samples,
and therefore having a consistently high rank on all these arrays).

| | Array | | | | | qPCR | | | |
|---|---|---|---|---|---|---|---|---|---|
| | CTCL | BDN | | | | CTCL | BDN | log2 | |
| | average | average | log2 | | | average | average | fold | |
| | log2 | log2 | fold | | P (Wilcox test | expression | expression | change | |
| microRNA | expression | expression | change | P (t-test) | on ranks) | (−ΔCt) | (−ΔCt) | (ΔΔCt) | P (t-test) |
| miR-203 | 6.11 | 6.88 | −0.77 | 2.50E−23 | 1.80E−20 | 5.2 | 8 | −2.8 | 4.00E−09 |
| miR-663b | 5.8 | 5.11 | 0.69 | 5.30E−17 | 3.70E−14 | 10.9 | 9.4 | 1.5 | 0.002 |
| miR-326 | 6.2 | 5.6 | 0.6 | 1.10E−15 | 8.10E−13 | 1.4 | −1 | 2.4 | 4.60E−08 |
| miR-711 | 6.13 | 5.43 | 0.7 | 2.00E−15 | 1.40E−12 | −6.3 | −4.5 | −1.8 | 0.02 |
| miR-205 | 6.25 | 7.34 | −1.1 | 5.00E−13 | 3.50E−10 | 6.2 | 9.4 | −3.2 | 3.30E−10 |

For miR-663b, the average expressions change in the same direction (increased in CTCL) for both microarray and qRT-PCR measurements, but the P-value is only 0.002 for the qRT-PCR measurements, which is a much lower significance than for miR-203, miR-205, and miR-326 (even when taking the lower number of samples tested into account).

The fluorescence amplification plot for miR-663b showed a biphasic behavior with two plateaus, which indicate that the TaqMan primers and probe does not perform well. Importantly miR-711 did not achieve significance in the qRT-PCR measurements.

### BIBLIOGRAPHY

Ambros (2003) RNA 9, 277-279
Ballabio E, Mitchell T, Kester M S van, et al. Microrna expression in sezary syndrome: identification, function, and diagnostic potential. Blood. 2010; 116:1105-13.
Burg G, Kempf W, Cozzio A, et al. Who/eortc classification of cutaneous lymphomas 2005: histological and molecular aspects. Journal of cutaneous pathology. 2005; 32:647-74.
Bustin S A, Nolan T. Pitfalls of quantitative real-time reverse-transcription polymerase chain reaction. Journal of biomolecular techniques. 2004; 15:155-66.
Chen J, Odenike O, Rowley J D. Leukaemogenesis: more than mutant genes. Nature reviews. Cancer. 2010; 10:23-36.
Cleveland W S, Grosse E, Shyu M J. Local regression models. Statistical Models in S. 1992; 309-376.
Dereure O, Levi E, Vonderheid E C, Kadin M E. Infrequent fas mutations but no bax or p53 mutations in early mycosis fungoides: a possible mechanism for the accumulation of malignant t lymphocytes in the skin. The Journal of investigative dermatology. 2002; 118:949-56.
Doorn R van, Kester M S van, Dijkman R, et al. Oncogenomic analysis of mycosis fungoides reveals major differences with sezary syndrome. Blood. 2009; 113:127-36.
Duda, Peter E. Hart, David G. Stork, “Pattern Classification”, Wiley Interscience, 2nd edition, 2001, pages 36-39
Fits L van der, Kester M S van, Qin Y, et al. Microrna-21 expression in cd4+ t cells is regulated by stat3 and is pathologically involved in sézary syndrome. The Journal of investigative dermatology. 2011; 131:762-8.
Foss F M, Zinzani P L, Vose J M, et al. Peripheral t-cell lymphoma. Blood. 2011;
Garzon R, Calin G a, Croce C M. Micrornas in cancer. Annual review of medicine. 2009; 60:167-79.
Girardi M, Heald P W, Wilson L D. The pathogenesis of mycosis fungoides. The New England journal of medicine. 2004; 350:1978-88.
Gjerdrum L M, Woetmann a, Odum N, et al. Foxp3+ regulatory t cells in cutaneous t-cell lymphomas: association with disease stage and survival. Leukemia 2007; 21:25.12-8.
Hoist L M, Kaczkowski B, Gniadecki R. Reproducible pattern of microrna in normal human skin. Experimental dermatology. 2010; 19:e201-5.
James Carpenter and John Bithell (2000) “Bootstrap confidence intervals: when, which, what? A practical guide for medical statisticians”. Statistics in Medicine 19, 1141-1164.
Kester M S van, Ballabio E, Benner M F, et al. Mirna expression profiling of mycosis fungoides. Molecular oncology. 2011; 5, 273-80.
Krejsgaard T, Vetter-Kauczok C S, Woetmann A, et al. Ectopic expression of B-lymphoid kinase in cutaneous T-cell lymphoma. Blood. 2009; 113:5896-904.
Lee B N, Duvic M, Tang C K, et al. Dysregulated synthesis of intracellular type 1 and type 2 cytokines by t cells of patients with cutaneous t-cell lymphoma. Clinical and diagnostic laboratory immunology. 1999; 6:79-84.
Narducci M G, Arcelli D, Picchio M C, et al. Microrna profiling reveals that mir-21, mir486 and mir-214 are upregulated and involved in cell survival in sézary syndrome. Cell death & disease. 2011; 2:e151.
Olsen E, Vonderheid E, Pimpinelli N, et al. Revisions to the staging and classification of mycosis fungoides and sezary syndrome: a proposal of the international society for cutaneous lymphomas (iscl) and the cutaneous lymphoma task force of the european organization of research and treatment of ca. Blood. 2007; 110:1713-22.
Ritchie M E, Silver J, Oshlack A, et al. A comparison of background correction methods for two-colour microarrays. Bioinformatics. 2007; 23:2700-7.
Rosen S T, Querfeld C. Primary cutaneous t-cell lymphomas. Hematology/the Education Program of the American Society of Hematology. American Society of Hematology. Education Program. 2006; 323-30, 513.
Rosenfeld N, Aharonov R, Meiri E, et al. Micrornas accurately identify cancer tissue origin. Nature biotechnology. 2008; 26:462-9.
Tibshirani R J, Efron B. Pre-validation and inference in microarrays. Statistical applications in genetics and molecular biology. 2002; 1:Article 1.
Trautinger F, Knobler R, Willemze R, et al. Eortc consensus recommendations for the treatment of mycosis fungoides/sézary syndrome. European journal of cancer. 2006; 42:1014-30.
Vandesompele J. De Preter K, Pattyn F, et al. Accurate normalization of real-time quantitative rt-per data by geometric averaging of multiple internal control genes. Genome biology. 2002; 3:RESEARCH0034.
Willemze R, Jaffe E S, Burg G, et al. Who-eortc classification for cutaneous lymphomas. Blood. 2005; 105:3768-85.

1. A method for classifying a test skin cell sample from an individual with an inflammatory skin disease as cutaneous lymphomas comprising:
detecting microRNA expression levels at least of one of miR-155 and miR-326, and at least of one of miR-203 and miR-205,
calculating a clinical score (S) of the test cell sample based on a dataset comprising the expression levels of said microRNAs, and
classifying the test cell sample as cutaneous lymphomas or not based on the value of the clinical score.

2. The method of claim 1 wherein the levels of microRNA miR-155 and at least one of miR-203 and miR-205 are detected and used to calculate a clinical score (S) of the test cell sample.

3. The method of claim 1 wherein the levels of microRNA miR-326 and at least one of miR-203 and miR-205 are detected and used to calculate a clinical score (S) of the test cell sample.

4. The method of claim 1 wherein the levels of microRNA miR-155 and miR-326 and at least one of miR-203 and miR-205 are detected and used to calculate a clinical score (S) of the test cell sample.

5. The method of claim 1 wherein the levels of microRNA miR-155, miR-326, miR-203 and miR-205 are detected and used to calculate a clinical score (S) of the test cell sample.

6. The method of claim 1 wherein the levels of microRNA miR-155, miR-326, miR-203, miR-205 and miR-663b are detected and used to calculate a clinical score (S) of the test cell sample.

7. The method of claim 1, wherein the cutaneous lymphoma is Cutaneous T-cell Lymphoma (CTCL).

8. The method of claim 1, wherein the clinical score is calculated as a ratio of the expression level of miR-155 and the average expression levels of miR-203 and/or miR-205.

9. The method of claim 1, wherein the clinical score is calculated as a ratio of the expression level of miR-326 and the average expression levels of miR-203 and/or miR-205.

10. The method of claim 1, wherein the clinical score is calculated as a ratio of the expression level of miR-155 and miR-326 relative to the average expression levels of miR-203 and/or miR-205.

11. The method of claim 1, wherein the expression levels of the microRNAs are determined by Q-PCR.

12. The method of claim 11, wherein the clinical score is calculated as follows:
S=X*C(miR-155)+Y*C(miR-205)+Z*C(miR-203)
wherein “C” is the threshold cycle value (Ct) or the crossing point value (Cp), and wherein X, Y, and Z are coefficients determined by linear regression, under the constraint that X+Y+Z=0.

13. The method of claim 11, wherein the clinical score is calculated as follows:
S=X*C(miR-326)+Y*C(miR-205)+Z*C(miR-203)
wherein “C” is the threshold cycle value (Ct) or the crossing point value (Cp), and wherein X, Y, and Z are coefficients determined by linear regression, under the constraint that X+Y+Z=0.

14. The method of claim 11, wherein the clinical score is calculated as follows:
S=C(miR-155)−C(miR-205)/2−C(miR-203)/2
wherein “C” is the threshold cycle value (Ct) or the crossing point value (Cp).

15. The method of claim 11, wherein the clinical score is calculated as follows:
S=C(miR-326)−C(miR-205)/2−C(miR-203)/2
wherein “C” is the threshold cycle value (Ct) or the crossing point value (Cp).

16. The method of claim 11, wherein the clinical score is calculated as follows:
S=C(miR-155)+C(miR-326)−C(miR-205)−C(miR-203)
wherein “C” is the threshold cycle value (Ct) or the crossing point value (Cp).

17. The method of claim 11, wherein the clinical score is calculated as follows:
S=C(miR-155)/3+C(miR-326)/3+C(miR-663b)/3−C(miR-205)/2−C(miR-203)/2
wherein “C” is the threshold cycle value (Ct) or the crossing point value (Cp).

18. The method of claim 14, wherein “C” is the crossing point value (Cp) and wherein when the clinical score “S” is lower than about 6.5, in particular lower than 6.0, the test indicates that the test skin cell sample is Cutaneous T-cell Lymphoma, and wherein when the clinical score “S” is higher than about 6.5, in particular higher than about 7.0, the test indicates that the test skin cell sample is benign.

19. The method of claim 11, wherein the clinical score is calculated as follows:
S=X*C(miR-155)+Y*C(miR-326)+Z*C(miR-663b)+W*C(miR-203)+Q*C(miR-205),
wherein “C” is the threshold cycle value (Ct) or the crossing point value (Cp), and wherein X, Y, Z, W, and Q are coefficients determined by linear regression, under the constraint that X+Y+Z+W+Q=0.

20. The method of claim 11, wherein the method of Q-PCR is UniRT.


SEQUENCE LISTING (49 sequences)

SEQ ID NO: 1 - DNA, 26 bp, Artificial Sequence
  Synthetic RT-primer L2TA3
ggtactagtttttttttttttttvnn

SEQ ID NO: 2 - DNA, 22 bp, Homo sapiens
  misc_feature (1)..(22): miR sequence let-7b
tgaggtagtaggttgtgtggtt

SEQ ID NO: 3 - DNA, 23 bp, Homo sapiens
  misc_feature (1)..(23): miR sequence miR-103
agcagcattgtacagggctatga

SEQ ID NO: 4 - DNA, 23 bp, Homo sapiens
  misc_feature (1)..(23): miR sequence miR-155
ttaatgctaatcgtgataggggt

SEQ ID NO: 5 - DNA, 22 bp, Homo sapiens
  misc_feature (1)..(22): miR sequence miR-184
tggacggagaactgataagggt

SEQ ID NO: 6 - DNA, 23 bp, Homo sapiens
  misc_feature (1)..(23): miR sequence miR-191
caacggaatcccaaaagcagctg

SEQ ID NO: 7 - DNA, 22 bp, Homo sapiens
  misc_feature (1)..(22): miR sequence miR-203
gtgaaatgtttaggaccactag

SEQ ID NO: 8 - DNA, 22 bp, Homo sapiens
  misc_feature (1)..(22): miR sequence miR-205
tccttcattccaccggagtctg

SEQ ID NO: 9 - DNA, 22 bp, Homo sapiens
  misc_feature (1)..(22): miR sequence miR-24
tggctcagttcagcaggaacag

SEQ ID NO: 10 - DNA, 22 bp, Homo sapiens
  misc_feature (1)..(22): miR sequence miR-299-5p
tggtttaccgtcccacatacat

SEQ ID NO: 11 - DNA, 20 bp, Homo sapiens
  misc_feature (1)..(20): miR sequence miR-326
cctctgggcccttcctccag

SEQ ID NO: 12 - DNA, 22 bp, Homo sapiens
  misc_feature (1)..(22): miR sequence miR-34b
caatcactaactccactgccat

SEQ ID NO: 13 - DNA, 23 bp, Homo sapiens
  misc_feature (1)..(23): miR sequence miR-423-5p
tgaggggcagagagcgagacttt

SEQ ID NO: 14 - DNA, 22 bp, Homo sapiens
  misc_feature (1)..(22): miR sequence miR-663b
ggtggcccggccgtgcctgagg

SEQ ID NO: 15 - DNA, 22 bp, Artificial Sequence
  Synthetic miR sequence
gggacccagggagagacgtaag

SEQ ID NO: 16 - DNA, 21 bp, Artificial Sequence
  Synthetic miR sequence
cttccgccccgccgggcgtcg

SEQ ID NO: 17 - DNA, 17 bp, Artificial Sequence
  Synthetic F primer
catgaggtagtaggttg

SEQ ID NO: 18 - DNA, 16 bp, Artificial Sequence
  Synthetic F primer
agcagcattgtacagg

SEQ ID NO: 19 - DNA, 20 bp, Artificial Sequence
  Synthetic F primer
gacttaatgctaatcgtgat

SEQ ID NO: 20 - DNA, 16 bp, Artificial Sequence
  Synthetic F primer
tggacggagaactgat

SEQ ID NO: 21 - DNA, 18 bp, Artificial Sequence
  Synthetic F primer
caacggaatcccaaaagc

SEQ ID NO: 22 - DNA, 18 bp, Artificial Sequence
  Synthetic F primer
gtgaaatgtttaggacca

SEQ ID NO: 23 - DNA, 17 bp, Artificial Sequence
  Synthetic F primer
tccttcattccaccgga

SEQ ID NO: 24 - DNA, 15 bp, Artificial Sequence
  Synthetic F primer
tggctcagttcagca

SEQ ID NO: 25 - DNA, 18 bp, Artificial Sequence
  Synthetic F primer
tggtttaccgtcccacat

SEQ ID NO: 26 - DNA, 16 bp, Artificial Sequence
  Synthetic F primer
cctctgggcccttcct

SEQ ID NO: 27 - DNA, 18 bp, Artificial Sequence
  Synthetic F primer
caatcactaactccactg

SEQ ID NO: 28 - DNA, 14 bp, Artificial Sequence
  Synthetic F primer
catgggcagagagc

SEQ ID NO: 29 - DNA, 12 bp, Artificial Sequence
  Synthetic F primer
ccggccgtgcct

SEQ ID NO: 30 - DNA, 15 bp, Artificial Sequence
  Synthetic F primer
agggacccagggaga

SEQ ID NO: 31 - DNA, 17 bp, Artificial Sequence
  Synthetic F primer
tatcttccgccccgccg

SEQ ID NO: 32 - DNA, 29 bp, Artificial Sequence
  Synthetic R primer
ggtactagtttttttttttttttaaccac

SEQ ID NO: 33 - DNA, 28 bp, Artificial Sequence
  Synthetic R primer
gtactagtttttttttttttttcatagc

SEQ ID NO: 34 - DNA, 29 bp, Artificial Sequence
  Synthetic R primer
gtactagtttttttttttttttaccccta

SEQ ID NO: 35 - DNA, 27 bp, Artificial Sequence
  Synthetic R primer
gtactagtttttttttttttttaccct

SEQ ID NO: 36 - DNA, 26 bp, Artificial Sequence
  Synthetic R primer
gtactagtttttttttttttttcagc

SEQ ID NO: 37 - DNA, 36 bp, Artificial Sequence
  Synthetic R primer
tgacacggaggtactagtttttttttttttttctag

SEQ ID NO: 38 - DNA, 28 bp, Artificial Sequence
  Synthetic R primer
gtactagtttttttttttttttcagact

SEQ ID NO: 39 - DNA, 37 bp, Artificial Sequence
  Synthetic R primer
tgacacggaggtactagttttttttttttttctgttc

SEQ ID NO: 40 - DNA, 30 bp, Artificial Sequence
  Synthetic R primer
gaggtactagtttttttttttttttatgta

SEQ ID NO: 41 - DNA, 27 bp, Artificial Sequence
  Synthetic R primer
gtactagtttttttttttttttctgga

SEQ ID NO: 42 - DNA, 28 bp, Artificial Sequence
  Synthetic R primer
ggtactagtttttttttttttttatggc

SEQ ID NO: 43 - DNA, 29 bp, Artificial Sequence
  Synthetic R primer
aggtactagttttttttttttttaaagtc

SEQ ID NO: 44 - DNA, 27 bp, Artificial Sequence
  Synthetic R primer
gtactagtttttttttttttttcctca

SEQ ID NO: 45 - DNA, 29 bp, Artificial Sequence
  Synthetic R primer
ggtactagtttttttttttttttcttacg

SEQ ID NO: 46 - DNA, 14 bp, Artificial Sequence
  Synthetic R primer
ttttttttcgacgc

SEQ ID NO: 47 - DNA, 10 bp, Artificial Sequence
  Synthetic PolyA tail of microRNA
aaaaaaaaaa

SEQ ID NO: 48 - DNA, 13 bp, Artificial Sequence
  Synthetic extension primer
ttttttttttvnn

SEQ ID NO: 49 - DNA, 10 bp, Artificial Sequence
  Synthetic DNA
tttttttttt